(19) United States
(12) Patent Application Publication
(10) Pub. No.: US 2009/0208986 A1
(43) Pub. Date: Aug. 20, 2009

(54) Method and kit for diagnosing acute myocardial infarction

(75) Inventors: Kyung-Hyun Cho, Suseong-gu (KR); Dong-Gu Shin, Suseong-gu (KR); Jae-Ryong Kim, Dalseo-gu (KR)

Correspondence Address: THE NATH LAW GROUP, 112 South West Street, Alexandria, VA, 22314, US

(73) Assignee: Industry-Academic Cooperation Foundation Yeungnam University, Gyeongsan-si (KR)

(21) Appl. No.: 12/320,164

(22) Filed: Jan. 21, 2009

(30) Foreign Application Priority Data
Jan. 22, 2008 (KR) 10-2008-0006829

(51) Int. Cl. G01N 33/53

(52) U.S. Cl. 435/7.92; 435/7.1; 435/4

(57) ABSTRACT

Disclosed herein are method and kit for diagnosing acute myocardial infarction which is capable of predicting and diagnosing whether coronary artery disease is proceed to myocardial infarction or not by using an increase in triglyceride level in high density lipoprotein (HDL) or low density lipoprotein (LDL), a decrease in cholesterol level in HDL, increase of interleukin-6, CETP (cholesteryl ester transfer protein) and apo (apolipoprotein) C-III as a biomarker that are characteristic changes occurred only in sera of myocardial infarction patients among patients with coronary artery disease.

### CROSS-REFERENCE TO RELATED PATENT APPLICATION

This non-provisional application claims priority under 35 U.S.C. § 119(a) on Korean Patent Application No. 10-2008-0006829 filed on Jan. 22, 2008, which is herein incorporated by reference.

### BACKGROUND OF THE INVENTION

1. Field of the Invention

The present invention relates to a method of diagnosing acute myocardial infarction comprising detecting the amount of serum interleukin-6, CETP, and apolipoprotein C-III from a serum specimen by using a diagnostic tool comprising an enzyme-linked immunosorbent assay (ELISA).

The present invention also relates to a diagnostic kit for acute myocardial infarction. In particular, the present invention relates to a diagnostic kit for acute myocardial infarction capable of predicting and diagnosing whether a patient's coronary artery disease can be proceeded to develop myocardial infarction judging by an increase in triglyceride level in high density lipoprotein (HDL) or low density lipoprotein (LDL), a decrease in cholesterol level in HDL, increase in interleukin-6, CETP (cholesteryl ester transfer protein) and apo (apolipoprotein) C-III as a biomarker which are characteristic changes occurred only in a serum of myocardial infarction patients among patients with coronary artery disease.

2. Description of the Background

Myocardial infarction is one of the most serious diseases which may lead to a sudden death in middle-aged adults, and its incidence rate has been on the increase. Although myocardial infarction and stable angina pectoris both belong to ischemic heart diseases, myocardial infarction differs from stable angina pectoris in terms of pathophysiology and clinical prognosis. Stable angina pectoris can be easily diagnosed by the presence of repetitive and characteristic chest pain or by examining a common exercise stress test. However, since myocardial infarction is developed by occlusive thrombus of coronary artery caused by sudden rupture of vulnerable plaques, it is impossible to diagnose it by using a conventional method, serum lipids, biomarkers and the like known in the art. Currently, the only way to detect the above vulnerable plaques is an invasive method of using vascular echocardiography. Because there is no biomarker capable of predicting such a change before the development of myocardial infarction, it is very difficult to diagnose the disease at its early stage.

Myocardial infarction is derived from coronary artery disease (e.g., angina pectoris) which is caused by narrowing the vascular lumen of the coronary artery as a lesion becomes larger by accumulating and growing atherosclerotic plaques which are necrotic cores of cholesterol and lymphocytes. Myocardial infarction is caused by thrombosis in which a fibrous cap of the lesion is easily ruptured due to its instability, and thereby, a thrombus, or blood clot, is formed, which results in complete clogging of the vascular lumen. Once myocardial infarction is developed, oxygen and nutrients cannot be supplied to the muscles below the clogged blood vessel, leading to the necrosis of myocardium. Therefore, myocardial infarction is initiated by atherosclerosis and developed for a long time, but it is different from angina pectoris. While, in case of angina pectoris, the lesion is stably formed and its fibrous cap is not easily ruptured, in case of myocardial infarction, the vulnerable plaques are formed characterized by easy formation and rupture of a lesion.

Especially, unlike American or European patients, myocardial infarction patients in Korea show a relatively low level of cholesterol in blood (240 mg/dL or less) and a relatively high level of HDL-cholesterol (40 mg/dL or more), and easy rupture of a lesion in spite of its small size, which often prevents an early diagnosis of myocardial infarction. Thus, there is a need for the development of a new biomarker capable of diagnosing myocardial infarction at its early stage. Therefore, it is very important to save a patient's life to predict and diagnose whether chronic coronary artery disease proceeds to acute myocardial infarction, whether a vulnerable atherosclerotic plaque being ruptured easily in spite of its small size is formed, and whether thus formed vulnerable plaque is easily ruptured.

Generally, the diagnosis of myocardial infarction is carried out by using an enzymatic method of measuring the increase in a biomarker protein in combination with an invasive method such as intravascular ultrasound (IVUS) or coronary angiography. Thus, since there is a need to anesthetize a patient for inserting a cardiac catheter, such a cumbersome makes impossible to early diagnosis of myocardial infarction before the occurrence of pain. Further, the currently used biomarkers of glutamic oxaloacetic transaminase (GOT), lactate dehydrogenase (LDH), creatine kinase MB (CK-MB), troponin I, and troponin T are not useful for early diagnosis of myocardial infarction but are only applicable after the development of myocardial infarction. Therefore, there is a need to develop a biomarker which is specific to myocardial infarction and can detect and predict the physiological change before the development of myocardial infarction.

There have been suggested several theories for investigating the causes of coronary artery disease. In particular, as oxidized LDL containing a large quantity of cholesterol and necrotic cores of macrophages (so-called foam cells) are accumulated in the vascular lumen of the coronary artery and brought up for a long time, arterial lesions or atherosclerotic plaques are formed. Thus formed atherosclerotic plaques become larger along with the progress of aging and immune inflammation, and finally, proceeds to angina pectoris with clogging the arterial lumen. When the atherosclerotic plaques are ruptured and thrombosis is occurred at the same time as the clogging of a blood vessel is getting worse, the myocardium located inferior to the clogged blood vessel fall into necrosis due to the lack of oxygen and nutrient supply. Since the currently available method for the diagnosis of myocardial infarction is to use a marker protein (troponin I and troponin T) indicating necrosis of myocardium as a postmortem examination, it is only possible to diagnose the disease after its development. However, unfortunately, there is no biomarker for detecting the formation of an atherosclerotic plaque and the extent of clogging the vascular lumen, and it is only possible to refer to the high level of serum cholesterol and LDL-cholesterol as a risk factor.

If the arterial lumen is clogged by 75% or more while increasing the size of an atherosclerotic plaque as coronary artery disease is progressing, a symptom of angina pectoris develops. Angina pectoris is divided into stable angina pectoris and unstable angina pectoris. Unlike stable angina pectoris in which a fibrous cap of the lesion is stably maintained, unstable angina pectoris is a fatal disease which has a high risk of rupturing an atherosclerotic plaque in spite of a small size. Therefore, extensive researches have been carried out on the identification of causes for converting a stable plaque into an unstable plaque, and the development of a biomarker capable of detecting such a change at an early stage can be effectively used for the early diagnosis of myocardial infarction.

It has been found that atherosclerosis known as a major cause of coronary artery disease becomes worse as an LDL-cholesterol level is increased, while it is improved as a HDL-cholesterol level is increased. Further, it has been also reported that the increase in lipoproteins containing a large quantity of triglycerides leads to deterioration of atherosclerosis (Circulation 2002; 106: 2137-2142), and the increase in inflammatory markers aggravates atherosclerosis and coronary artery disease. However, there is no report on a biomarker capable of specifically discriminating the risk of myocardial infarction and angina pectoris among the coronary artery diseases.

### SUMMARY OF THE INVENTION

Coronary artery disease is one of the most chronic diseases and is difficult to diagnose at its early stage among cardiovascular diseases. Of them, ischemic myocardial infarction has been one of the most fatal diseases threatening a patient's life but there has been no biomarker available for its early diagnosis.

Since the conventional methods for the diagnosis of myocardial infarction are carried out by using a biomarker applicable only after its development or by using electrocardiography or invasive coronary arthrography, they have poor patients' compliance and are not cost effective. The present inventors have therefore endeavored to develop a diagnostic kit for simply and rapidly predicting the development of myocardial infarction, and compared various kinds of lipids and lipoprotein markers in a serum of angina pectoris patients with those of myocardial infarction patients among patients with coronary artery disease. As a result, the present inventors have found that an increase in triglyceride level in high density lipoprotein (HDL) or low density lipoprotein (LDL), a decrease in cholesterol level in HDL, increase of interleukin-6, CETP (cholesteryl ester transfer protein) and apo (apolipoprotein) C-III can be used as a biomarker capable of discriminating angina pectoris from myocardial infarction.

Therefore, embodiments of the present invention have been made in view of the above problems of the prior art, and it is one objective of embodiments of the present invention to provide a diagnostic method and a diagnostic kit for easily predicting the conversion into myocardial infarction.

In accordance with one aspect of embodiments of the present invention for achieving the above objective, there is provided a diagnostic method and a diagnostic kit for acute myocardial infarction by using the increase in triglyceride level in high density lipoprotein (HDL) or low density lipoprotein (LDL), the decrease in cholesterol level in HDL, increase of interleukin-6, CETP (cholesteryl ester transfer protein) and apo (apolipoprotein) C-III as a biomarker.

### BRIEF DESCRIPTION OF THE DRAWINGS

The above and other objects, features and other advantages of embodiments of the present invention will be more clearly understood from the following detailed description taken in conjunction with the accompanying drawings, in which:

FIG. 1 shows a graph showing the comparison of paraoxonase activity between myocardial infarction patients and angina pectoris patients;

FIG. 2 shows a graph showing the comparison of lecithin cholesterol acyltransferase activity between myocardial infarction patients and angina pectoris patients;

FIG. 3 shows photographs illustrating a CETP level detected in each lipoprotein fraction of myocardial infarction patients and angina pectoris patients;

FIG. 4 shows photographs illustrating an expression pattern of apoC-III in each lipoprotein fraction of myocardial infarction patients and angina pectoris patients;

FIG. 5 shows a graph showing the comparison of the extent of LDL oxidation between myocardial infarction patients and angina pectoris patients; and

FIG. 6 shows a photograph illustrating the comparison of LDL mobility between myocardial infarction and angina pectoris patients.

### DETAILED DESCRIPTION OF THE PREFERRED EMBODIMENTS

Hereinafter, embodiments of the present invention will be explained in more detail with reference to the accompanying drawings.

The present invention relates to a method of diagnosing acute myocardial infarction comprising detecting the amount of interleukin-6, CETP, and apoliopoproteinC-III from a serum specimen by using a diagnostic tool comprising an enzyme-linked immunosorbent assay (ELISA).

The present invention also relates to a diagnostic kit for acute myocardial infarction which is capable of predicting and deciding whether coronary artery disease proceeds to myocardial infarction or not by using an increase in triglyceride level in high density lipoprotein (HDL) or low density lipoprotein (LDL), a decrease in cholesterol level in HDL, increase of interleukin-6, CETP (cholesteryl ester transfer protein) and apo (apolipoprotein) C-III as a biomarker that are characteristic changes occurred only in a serum of myocardial infarction patients among patients with coronary artery disease.

In order to develop a new biomarker specific to myocardial infarction, the present invention has studied for patients who attended to a division of cardiology, department of internal medicine, Yeungnam University Medical Center (Korea) suffering from chest pain and ache during the period from January, 2007 to June, 2007. As a result of examining the patients with coronary angiography and serological tests, the male patients with angina pectoris (n=14) and male patients with myocardial infarction (n=6) were selected and showed written consent to participate in the study. After blood samples were taken from each patient, blood and lipoprotein fractions were analyzed and compared each other so as to discover a biomarker and physiological changes specific for the myocardial infarction patients.

The experimental methods used in this study are as follows.

1. Comparison of enzymatic features and lipid composition in a serum

2. Analysis of an inflammatory biomarker in a serum

3. Separation of lipoprotein fractions from a serum by using ultracentrifugation

4. Analysis of lipid composition in lipoprotein fractions (VLDL, LDL, HDL₂, HDL₃)

5. Comparison of enzyme activity of lipoprotein fractions. Analysis of LCAT (lecithin:cholesterol acyltransferase), CETP (cholesteryl ester transfer protein), PON (paraoxonase), Lp-PLA2 (lipoprotein-associated phospholipase A2) activities

6. Analysis of protein expression of lipoprotein fractions by using Western blotting

According to these experiments, the present inventors have confirmed that an increase in triglyceride level in high density lipoprotein (HDL) or a low density lipoprotein (LDL) fraction, a decrease in cholesterol level in HDL, increase of interleukin-6, CETP and apo C-III can be used as a biomarker for discriminating the diagnosis of myocardial infarction from that of angina pectoris. That is, the present invention relates to a diagnostic kit for acute myocardial infarction which detects the amount of interleukin-6 in a serum specimen by using an enzyme-linked immunosorbent assay (ELISA).

In particular, the myocardial infarction patients show 10 pg/ml or more of interleukin-6, being discriminated from the amount of interleukin-6 in the angina pectoris patients.

Further, during the fractionation of lipoproteins in a serum, the residual fraction obtained by ultracentrifugation while adjusting the density to d<1.019 g/mL was adjusted its density to 1.019<d<1.063 (g/mL) and subjected to ultracentrifugation, to thereby separate an LDL fraction. The residual fraction was adjusted its density to 1.063<d<1.125 (g/mL) and subjected to ultracentrifugation, to thereby separate a HDL₂ fraction. The residual fraction was adjusted its density to 1.125<d<1.225 (g/mL) and subjected to ultracentrifugation, to thereby separate a HDL₃ fraction.

Thus obtained lipoprotein fractions are subjected to electrophoresis and the expression of CETP (cholesteryl ester transfer protein) or apoC-III in the above fractions is examined. As a result, it has been confirmed that CETP and apoC-III can be effectively used as a biomarker for the diagnosis of myocardial infarction. It is preferable to use the LDL or HDL₃ fraction among the lipoprotein fractions for the diagnosis of myocardial infarction.

Therefore, the present invention relates to a diagnostic kit for acute myocardial infarction which comprises an antibody specifically binding to CETP (cholesteryl ester transfer protein) or apoC-III.

The expression of the above lipoproteins can be analyzed by Western blotting. There is no limitation to the kind of an antibody specifically binding to CETP so long as it is capable of specifically binding to an amino terminus or a carboxyl terminus of CETP and can be commercially available as a monoclonal antibody or a polyclonal antibody. In a preferred embodiment of the present invention, a polyclonal antibody Ab19012-100 (ABcam Inc., England) is used as an antibody specifically binding to CETP. Further, there is no limitation to the kind of an antibody specifically binding to apoC-III so long as it is capable of specifically binding to an amino terminus or a carboxyl terminus of apoC-III and can be commercially available as a monoclonal antibody or a polyclonal antibody. In a preferred embodiment of the present invention, a polyclonal antibody AB821 (Chemicon Inc., USA) is used as an antibody specifically binding to apoC-III.

Further, the present invention has confirmed that an increase in triglyceride level in a high density lipoprotein (HDL) fraction of a serum specimen or a low density lipoprotein (LDL) fraction thereof, and a decrease in cholesterol level in a HDL fraction thereof can be used as a biomarker for the diagnosis of myocardial infarction, and therefore, includes a diagnostic kit for acute myocardial infarction using the above biomarkers.

A triglyceride level in the HDL₂ fraction derived from myocardial infarction patients is measured in the range of 62-80 mg/dL by using a serum automated analyzer (Electa biochemical analyzer, Italy) or a kit for measuring a triglyceride level (TG-S, Asan Pharmaceutical, AM157S-K), which is significantly higher than that in the HDL₂ fraction derived from the angina pectoris patients, one of patients with coronary artery disease.

Further, a triglyceride level in the LDL fraction derived from myocardial infarction patient is measured in the range of 185-305 mg/dL by using a serum automated analyzer (Electa biochemical analyzer, Italy) or a kit for measuring a triglyceride level (TG-S, Asan Pharmaceutical, AM157S-K), which is significantly higher than that in the LDL fraction derived from the angina pectoris patients, one of patients with coronary artery disease.

Further, a cholesterol level in the HDL₂ fraction derived from myocardial infarction patients is measured in the range of 46-58 mg/dL by using a serum automated analyzer (Electa biochemical analyzer, Italy) or a kit for measuring a total cholesterol level (Total cholesterol, Asan Pharmaceutical, AM202-K), which is significantly lower than that in the HDL₂ fraction derived from the angina pectoris patients, one of patients with coronary artery disease.

Therefore, since the present invention has developed biomarkers specific for patients with myocardial infarction which is fatal and difficult to obtain early diagnosis among several types of coronary artery diseases, it is possible to easily predict the conversion into myocardial infarction, exactly diagnose the same at its early stage, minimize patients' inconvenience due to the use of an invasive method, and thereby, expect significant economic and social contribution effects.

Embodiments of the present invention will now be described in more detail with reference to the following examples. However, the examples are given for the purpose of illustration and are not to be construed as limiting the scope of the invention.

### Test Example 1

### Lipid Profile Analysis of Patients' Sera

A blood sample was taken from each patient by using a vacutainer tube containing EDTA (Beckton-Dickinson, Franklin Lakes, N.J., USA), and a serum was separated from the blood sample by using a low-speed centrifuge (6000 g). Profiles of blood lipids and major biomarkers for the patients were analyzed by using a Chemistry analyzer AU4500 (Olympus, Japan) and are shown in Table 1.

TABLE 1
Comparison of serum lipid profiles between the angina pectoris
patients and the myocardial infarction patients

| | | myocardial infarction |
|---|---|---|
| | angina pectoris patients | patients |
| Group | (n = 17) | (n = 6) |
| Age (yr) | 60.6 ± 9.8 | 51.5 ± 10.8 |
| BMI (kg/m²) | 23.5 ± 2.5 | 23.9 ± 2.3 |
| TC (mg/dL) | 157 ± 33 | 208 ± 20* |
| LDL-C (mg/dL) | 106 ± 30 | 118 ± 35 |
| HDL-C (mg/dL) | 48 ± 8 | 54 ± 13 |
| % HDL-C | 30 ± 2 | 25 ± 3* |
| TG (mg/dL) | 122 ± 39 | 175 ± 49** |
| Glucose (mg/dL) | 149 ± 53 | 121 ± 18 |

*P < 0.05,
**p < 0.01
BMI: body mass index;
TC: total cholesterol;
LDL-C: low-density lipoprotein-cholesterol;
HDL-C: high-density lipoprotein-cholesterol;
TG: triglyceride

According to the results shown in Table 1, there was no meaningful difference in a body mass index (BMI value), blood LDL-cholesterol (LDL-C), HDL-cholesterol (HDL-C) and glucose levels between the myocardial infarction patients and the angina pectoris patients. However, total blood cholesterol and triglyceride levels of the myocardial infarction patients were increased by 24% and 30%, respectively, as compared with the angina pectoris patients. On the other hand, in case of the myocardial infarction patients, the ratio of HDL-cholesterol in the total cholesterol was decreased from 30±2% to 25±3%. However, the total cholesterol level in the myocardial infarction patients did not exceed the recommended criteria for hypercholesterolemia (serum level 240 mg/dL or more) as provided by NCEP (National cholesterol education program)-Adult treatment panel (ATP)-III), as an advice of American Heart Association. These results suggest that there is a limit to use the serum cholesterol, LDL-cholesterol or HDL-cholesterol level as a biomarker for the diagnosis of myocardial infarction. The triglyceride level of the myocardial infarction patients was higher (175±49 mg/dL) than the recommended criteria (150 mg/dL) of NCEP-ATP-III. Further, there was no meaningful difference in glucose level between the myocardial infarction patients and the angina pectoris patients.

### Test Example 2

### Analysis of the Amount of Proteins and Enzymes in Patients' Sera

The amount of inflammatory marker proteins and hepatic maker proteins in patients' sera was measured by using an automated serum analyzer and an ELISA kit.

As a result of quantifying with the automated serum analyzer, the myocardial infarction patients showed several-fold higher GOT and LDH levels than the angina pectoris patients, and showed several hundred-fold higher Troponin I (Dimension Expand Inc., Dade Behring, Deerfield, Ill., USA), Troponin T (Elecsys 2010, Hitachi, Tokyo, Japan), and CK-MB (Dimension Expand Inc., Dade Behring, Deerfield, Ill., USA) levels than the angina pectoris patients, which comes under the typical characteristics of ischemic myocardial necrosis.

As a result of measuring a serum level of MPO (myeloperoxidase), one of inflammatory markers being in the spotlight recently, with an ELISA kit (Immunology Consultants Lab, Cat #E-80PX, Newberg, Oreg., USA), the MPO level was increased both in the myocardial infarction patients and the angina pectoris patients by 2-fold or more, which suggests that MPO plays an important role in the development and progression of coronary artery disease. However, there was no unique increase as a biomarker for the diagnosis of myocardial infarction.

As a result of measuring a serum level of SAA (serum amyloid A), one of acute inflammatory markers, with an ELISA kit (Biosource cat #KNA0012, Camarillo, Calif., USA), both in the myocardial infarction patients and the angina pectoris patients, the SAA level was detected in the range of 50 to 60 mg/L, which was several-fold higher than the normal range (6-15 mg/dL). These results suggest that SAA can be used as a biomarker for coronary artery disease, but it is also unable to use a biomarker specific for myocardial infarction.

### Example 1

### Detection of the Amount of Interleukin (IL)-6

The amount of interleukin (IL)-6 as an inflammatory marker different from that of Test Example 2 was detected by using an ELISA kit (R&D systems, human quantikine IL-6, cat# D6050).

To a 96-well plate in which a bottom was coated with an IL-6 antibody (primary antibody) was added a mixture of a serum specimen 0.1 mL and a reaction reagent 0.1 mL, and the well plate was reacted for 2 hours. After the reaction was completed, the well plate was washed with a washing reagent to remove unbound proteins of the serum specimen. A secondary antibody specifically binding to the primary antibody (fluorescent enzyme coupled antibody) 0.2 mL was added to the well plate, followed by reacting for 2 hours. After the reaction was completed, the well plate was washed with the washing reagent to remove the unbound secondary antibody. After the washing, a substrate solution for the fluorescent enzyme was added to the well plate and color development was induced for 20 minutes. An absorbance of each well was detected at 450 nm and compared with that of a reference standard to quantify the amount of IL-6. As a result, the angina pectoris patients (6.6±5.4 pg/mL) showed higher amount of IL-6 than the myocardial infarction patients (15.6±4.1 pg/mL), which suggests that the increase in IL-6 level is specific for myocardial infarction. As compared with the fact that the IL-6 level of a normal person is lower than 3 pg/mL, it has been considered that an immune response of the patients with coronary artery disease is further increased in the myocardial infarction patients.

### Test Example 3

### Quantitation of Serum Uric Acid

Uric acid in patients' serum was determined according to a Caraway method [Caraway WT. Determination of uric acid in serum by a carbonate method. Am. J. Clin. Pathol. 1955; 25: 840-845.] and the results were compared with each other. As a result, there was no meaningful difference between the myocardial infarction patients and the angina pectoris patients, and both groups of the myocardial infarction and angina pectoris patients showed a normal range of uric acid level (2-7 mg/dL).

TABLE 2
Comparison of serum biomarkers between the angina pectoris
patients and the myocardial infarction patients

| | angina pectoris | myocardial infarction patients |
|---|---|---|
| Group | patients (n = 17) | (n = 6) |
| hsCRP (mg/dL) | 0.7 ± 1.3 | 0.8 ± 1.1 |
| MPO (ng/mL) | 9.6 ± 4.8 | 9.1 ± 4.6 |
| LDH (U/L) | 350 ± 66 | 728 ± 249* |
| SAA (mg/mL) | 60 ± 60 | 52 ± 58 |
| IL-6 (pg/mL) | 6.6 ± 5.4 | 15.6 ± 4.1* |
| GOT (U/L) | 23 ± 5 | 117 ± 103** |
| GPT (U/L) | 28 ± 13 | 37 ± 29 |
| Uric acid | 8.2 ± 2.3 | 5.8 ± 1.7 |
| (mg/dL) | | |
| Troponin I | 0.04 ± 0.03 | 30 ± 13** |
| (ng/mL) | | |
| Troponin T | 0.01 ± 0.01 | 4.9 ± 2.8** |
| (ng/mL) | | |
| CK-MB (ng/mL) | 1.2 ± 0.5 | 176 ± 156** |

hsCRP: high sensitivity C-reactive protein
LDH: lactate dehydrogenase
GOT: glutamic oxaloacetic transaminase
GPT: gamma-glutamic pyruvic transaminase
CK-MB: creatine kinase-MB fraction
*p < 0.05,
**p < 0.01

### Example 2

### Change in Lipid-Protein Composition of Lipoproteins

The density of all patients' sera samples was normalized by using sodium chloride (NaCl) or potassium bromide (KBr) according to a method described in the preceding document of the present inventors [Eur. J. Clin. Invest. 2007; 37: 249-256], and each sample was subjected to ultracentrifugation (100,000 g, Hitachi Co, Himac CP90a, Tokyo, Japan) for 24 hours. In order to separate a VLDL (very low density lipoprotein) fraction, the density of the serum was adjusted to d<1.019 g/mL, and the serum was subjected to ultracentrifugation. The residual fraction was adjusted its density to 1.019<d<1.063 (g/mL) and subjected to ultracentrifugation, to thereby separate an LDL fraction. The residual fraction was adjusted its density to 1.063<d<1.125 (g/mL) and subjected to ultracentrifugation, to thereby separate a HDL₂ fraction. After that, the residual fraction was adjusted its density to 1.125<d<1.225 (g/mL) and subjected to ultracentrifugation, to thereby separate a HDL₃ fraction.

Each lipoprotein fraction separated above was subjected to dialysis for 24 hours by using a PBS buffer to remove salts, and the amount of cholesterol, triglyceride and protein was quantified. As shown in Table 3, there was no change in the amount of cholesterol, triglyceride and protein in the VLDL fraction. However, in the LDL fraction, the significantly increased amount of cholesterol, triglyceride and protein was observed in the myocardial infarction patients group. In the HDL₂ fraction of the myocardial infarction patients, while the cholesterol level was decreased, the TG level was increased. In the HDL₃ fraction, the cholesterol level of the myocardial infarction patients was also decreased, and there was no meaningful difference in the triglyceride level.

As illustrated in the present invention (Example 1, Table 1), it has been found that the TG increase in a serum is an important characteristic of the myocardial infarction patients, and the results coincide with the recent report that the TG increase in a serum is proportion to the increase in coronary artery disease [J. Am. Med Assoc. 2007; 298: 336-338, J. Am. Med Assoc. 2007; 298: 299-308; J. Am. Med Assoc. 2007; 309-316]. Further, these results agree to the report that the risk of cardiovascular disease in the Asia-Pacific region is in proportion to the increase in serum triglyceride [Circulation 2004; 110: 2678-2686].

The present invention has demonstrated that the triglyceride level in a serum is significantly increased in the myocardial infarction patients among the patients with coronary artery disease, and in the serum of the myocardial infarction patients, and the TG level is specifically increased in LDL and HDL, and suggest the application of these results to the manufacture of a diagnostic kit. It has been well-known in the art that the increase in serum TG makes worse vascular inflammation proportionally [Circ. Res. 2007; 100: 381-390, Circ. Res. 2007; 100: 299-301], coronary artery disease is originated from arteriosclerosis, and aggravation of hyperlipidemia and immune inflammatory response plays an important role in the development of arteriosclerosis. It has been suggested that the change of desirable function of HDL that alleviates and suppresses the immune response of myocardial infarction into undesirable one is correlated with the decrease in HDL-cholesterol and increase in HDL-TG. Therefore, the decrease in HDL-cholesterol or increase in HDL-TG can be used as a biomarker for the diagnosis of myocardial infarction.

TABLE 3
Lipid and protein composition of lipoproteins from patients

| | angina pectoris patients (n = 10) | | | myocardial infarction patients (n = 6) | | |
|---|---|---|---|---|---|---|
| | cholesterol | triglyceride | protein | cholesterol | triglyceride | protein |
| Group | (mg/dL) | (mg/dL) | (g/dL) | (mg/dL) | (mg/dL) | (g/dL) |
| VLDL | 143 ± 31 | 345 ± 84 | 0.29 ± 0.07 | 141 ± 35 | 330 ± 98 | 0.36 ± 0.3 |
| (mg | (0.49 ± 0.1) | (1.19 ± 0.29) | (1) | (0.39 ± 0.09) | (0.91 ± 0.27) | (1) |
| lipid/mg | | | | | | |
| of | | | | | | |
| protein) | | | | | | |
| LDL (mg | 1071 ± 359 | 169 ± 57 | 0.62 ± 0.13 | 1563 ± 453* | 245 ± 60* | 0.88 ± 0.29 |
| lipid/mg | (1.72 ± 0.57) | (0.27 ± 0.09) | (1) | (1.77 ± 0.5) | (0.27 ± 0.06) | (1) |
| of | | | | | | |
| protein) | | | | | | |
| HDL₂ (mg | 70 ± 16 | 43 ± 274 | 0.19 ± 0.03 | 52 ± 6 | 71 ± 9* | 0.18 ± 0.04 |
| lipid/mg | (0.36 ± 0.08) | (0.22 ± 0.14) | (1) | (0.28 ± 0.03) | (0.39 ± 0.05) | (1) |
| of | | | | | | |
| protein) | | | | | | |
| HDL₃ (mg | 71 ± 23 | 25 ± 14 | 0.39 ± 0.1 | 54 ± 12 | 17 ± 7 | 0.34 ± 0.05 |
| lipid/mg | (0.18 ± 0.05) | (0.06 ± 0.03) | (1) | (0.15 ± 0.03) | (0.05 ± 0.02) | |
| of | | | | | | |
| protein) | | | | | | |

*p < 0.05

### Test Example 5

### Analysis of Paraoxonase Activity in a Serum, HDL₂ and HDL₃ Fractions

Paraoxonase activity was analyzed using patients' sera of 10 μL, or HDL₂ (1 mg/ml) and HDL₃ fractions (2 mg/mL) according to methods described by Eckerson et al. [Eckerson H W, Wyte C M, La Du B N. The human serum paraoxonase/arylesterase polymorphism. Am. J. Hum. Genet. 1983; 35: 1126-1138] and in the preceding document of the present inventors [Cho K H, Park J E, Kim Y O, Choi I, J I Kim, J R Kim. (2008) The function, composition, and particle size of high-density lipoprotein were severely impaired in an oliguric phase of hemorrhagic fever with renal syndrome [Clin. Biochem. 2008; 41: 56-64].

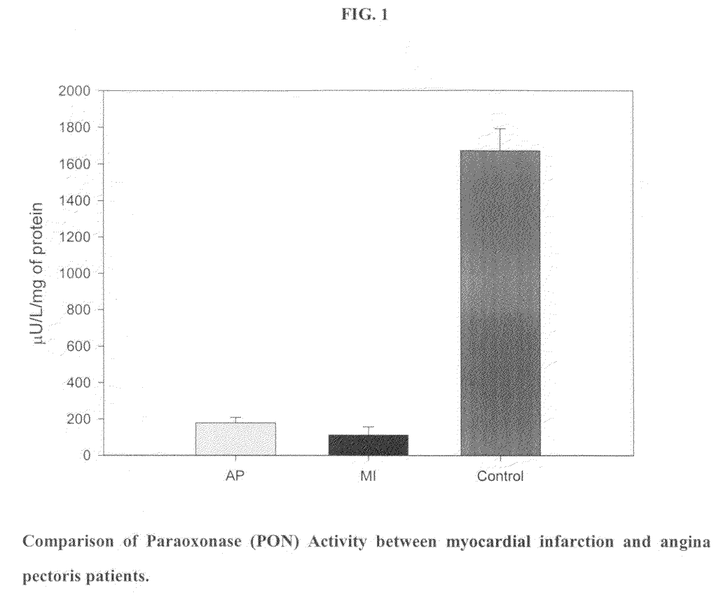

More specifically, the procedure used to generate the data depicted in FIG. 1 was as follows. 10 μL of serum and 200 μL of substrate (paraoxon-ethyl) were incubated in 90 mM Tris-HCl/3.6 mM NaCl/2 mM CaCl₂, pH 8.5. A PON-1 activity of 1 U/L is defined as 1 mmol of p-nitrophenol formed per minute. The molar extinction coefficient of p-nitrophenol is 17,000 M-1, cm-1.

As shown in FIG. 1, according to the results of measuring the paraoxonase activity in the HDL₃ fraction, the activity was significantly reduced in both myocardial infarction and angina pectoris patients, and thereby, antioxidant activity of HDL was also remarkably reduced. However, since the paraoxonase activity was significantly reduced in all coronary artery patients groups, there is a limit to use the paraoxonase activity as a biomarker for the diagnosis of myocardial infarction. In FIG. 1, the error bars indicate the standard deviation (SD) from three independent experiments conducted with duplicate samples.

### Test Example 6

### Change in LCAT Activity in a HDL₃ Fraction

LCAT activity was measured in a HDL₃ fraction of patients as an enzyme source by using an isotope-labeled [¹⁴C]-cholesterol according to a method described in the preceding document of the present inventors [J. Lipid Res. 2005; 46: 589-596].

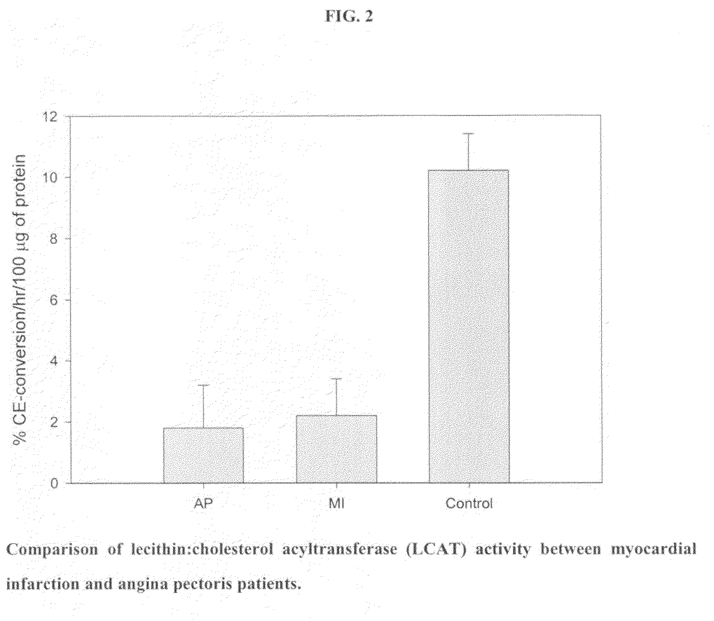

The procedure used to generate the data depicted in FIG. 2 was as follows. 50 μL of each serum sample and reconstituted HDL (rHDL) containing ¹⁴C-cholesterol were utilized as the LCAT source and substrate, respectively. Esterification was allowed for 1 hour at 37° C.

According to the result shown in FIG. 2, the LCAT activity in all coronary artery patients groups was reduced as compared with a control group. From these results, it has been found that the LCAT activity is related to the increase in HDL-triglyceride, and LCAT responsible for antioxidant function of HDL lacks in the patients with coronary artery disease. Therefore, there is a limitation to use the decrease in LCAT activity as a biomarker specific for the diagnosis of myocardial infarction.

### Example 7

### Measurement of CETP Activity

According to the previous journal of the present inventors [Biochim. Biophys. Acta 1391: 133-144], recombinant HDL containing [³H]-cholesteryl oleate ([³H]-CE-reconstituted HDL) was synthesized and used as a CE-donor. Each of a serum, VLDL, LDL, HDL₂, and HDL₃ was treated with a CETP protein source, and reacted with a human LDL (2 mg/mL) used as a CE-receptor for 6 hours. After the reaction was completed, the receptor LDL was separated and subjected to scintillation counting to measure the amount of radioactivity. Thus measured amount was used to calculate the activity of transferring from the CE-donor to the CE-receptor.

As shown in Table 4, the patients with coronary artery disease showed a higher CETP activity than a control group. However, there was no meaningful difference in CETP activity between the angina pectoris patients and the myocardial infarction patients. Interestingly, the LDL-CETP activity in the patients with coronary artery disease was 2-fold or more higher than that of the control group, but the HDL₃-CETP activity thereof was lower than that of the control group. These results suggest that there is a correlation between the increase in HDL-TG and the reduction in HDL function in the myocardial infarction patients.

TABLE 4
Comparison of CETP activity between a serum and a
lipoprotein fraction

| | % CE transfer/mg of protein | | |
|---|---|---|---|
| | angina pectoris | myocardial | control |
| Group | (n = 10) | infarction (n = 6) | (n = 5) |
| HDL₂ | 13.5 ± 12.1 | 8.9 ± 4.7 | 8.0 ± 4.6 |
| HDL₃ | 14.6 ± 4.7* | 15.4 ± 3.6* | 21.5 ± 2.5 |
| LDL | 7.6 ± 3.3* | 7.3 ± 2.5* | 2.7 ± 0.1 |
| VLDL | 3.0 ± 0.4 | 2.7 ± 0.6 | — |
| Serum | 29.7 ± 8.0 | 31.8 ± 6.3 | 24 ± 3.3 |

*p < 0.05

### Example 3

### Expression Analysis of CETP Protein

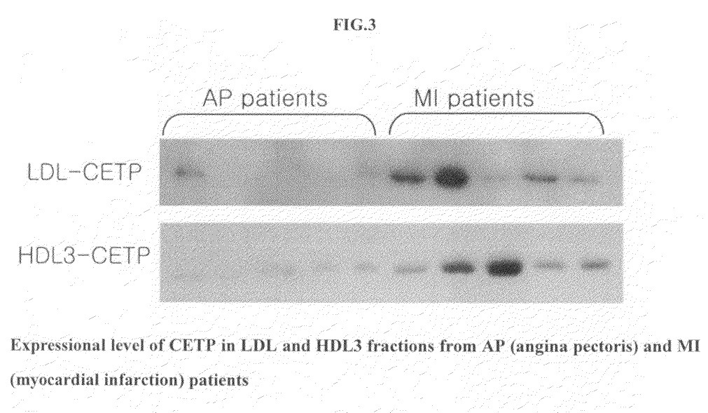

Each lipoprotein fraction obtained from the patients groups was diluted to a same concentration (protein-based: 2 mg/mL) and subjected to 10% SDS-PAGE. The lipoproteins loaded on the gel were transferred to a PVDF (polyvinylidene fluoride) membrane according to a method described by Towbin et al. [J. Immunol. Methods 1984; 72: 313-340]. In order to compare the relative expression amount of CETP among the proteins transferred onto the PVDF membrane, the PVDF membrane was subjected to Western blotting by using a CETP-polyclonal antibody (Abcam cat# ab19012) as a primary antibody (diluted by 1:1000) and a HRP (horse radish peroxidase)-conjugated secondary antibody specific for the primary antibody (Santa Cruz, SC2004) (diluted by 1:2000). As shown in FIG. 3, it has been found that the expression amount of CETP is relatively increased in the LDL and HDL₃ fractions.

These results demonstrate that the relative expression amount of CETP is specifically increased only in the myocardial infarction patients, and thereby, CETP can be used as a biomarker for the diagnosis and prediction of myocardial infarction.

### Example 4

### Expression Analysis of Serum apoC-III

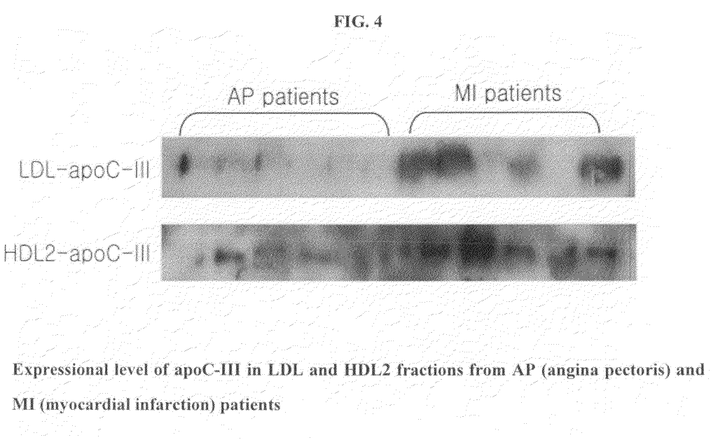

Each lipoprotein fraction obtained from the patients groups was diluted to a same concentration (protein-based: 2 mg/mL) and subjected to 15% SDS-PAGE. The lipoproteins loaded on the gel were transferred to a PVDF (polyvinylidene fluoride) membrane according to a method described by Towbin et al. [J. Immunol. Methods 1984; 72: 313-340]. In order to compare the relative amount of apoC-III expressed among the proteins transferred onto the PVDF membrane, the PVDF membrane was subjected to Western blotting by using an apoC-III-polyclonal antibody (Chemicon AB821) as a primary antibody (diluted by 1:1000) and a HRP (horse radish peroxidase)-conjugated secondary antibody specific for the primary antibody (Santa Cruz, SC2020) (diluted by 1:2000). As shown in FIG. 4, it has been found that apoC-III is specifically expressed in the LDL and HDL₂ fractions of the myocardial infarction patients.

### Example 5

### Comparison of LDL Oxidation

In order to examine the extent of LDL oxidation in each patient group, the amount of MDA (Malondialdehyde) as an oxidation product was measured in LDL at a same concentration (protein-based: 2 mg/mL) according to a method described by Blois [Blois M S. Antioxidant determinations by the use of a stable free radical. Nature 1958; 181: 1199-1200].

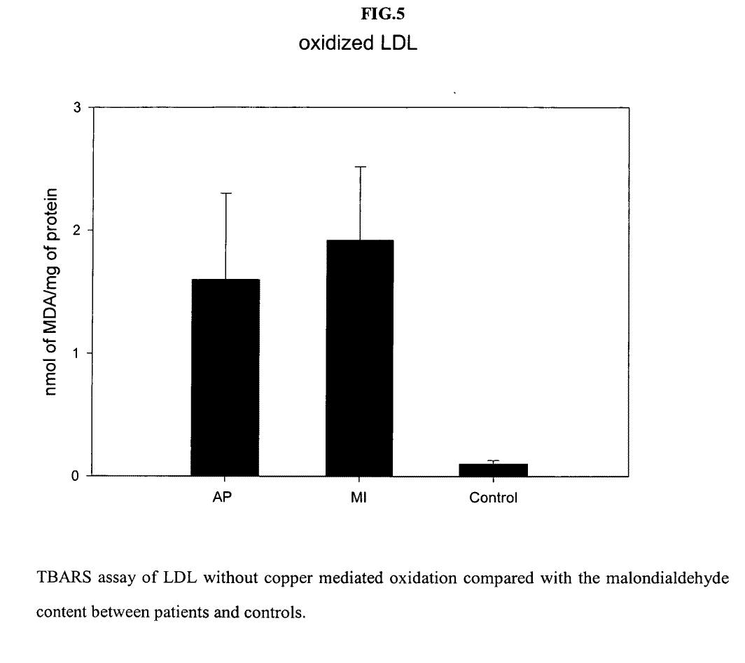

As shown in FIG. 5, both the myocardial infarction patients and the angina pectoris patients showed considerably high oxidation rate of LDL as compared with a control group, and in particular, the myocardial infarction patients showed the highest increase in LDL oxidation (increased by 92% as compared with the control group).

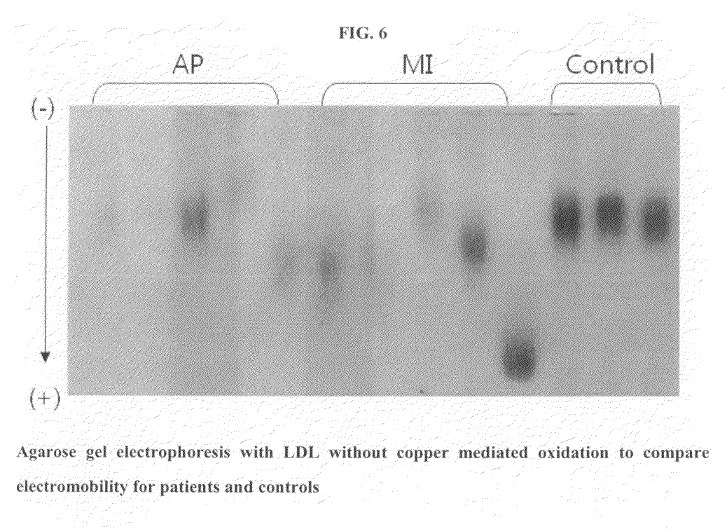

The LDL fractions of the above two groups were diluted to a same concentration (2 mg/mL) and subjected to agarose gel electrophoresis to examine mobility. As shown in FIG. 6, the myocardial infarction patients group showed faster mobility than the angina pectoris patients group. These results have coincided with the previous fact that the net electrical charge of LDL is increased as its oxidation is conducted, and thereby, its mobility on a gel becomes faster. Further, the above results have agreed to the increase in inflammatory marker enzyme (GOT) or cytokine (IL-6) of the myocardial infarction patients.

Although the preferred embodiments of the present invention have been disclosed for illustrative purpose, those skilled in the art will appreciate that various modifications, additions and substitutions are possible, without departing from the scope and spirit of the invention as disclosed in the accompanying claims.

1. A method of diagnosing acute myocardial infarction comprising detecting the amount of interleukin-6, cholesteryl ester transfer protein (CETP), and apolipoprotein C-III from a serum specimen by using a diagnostic tool comprising enzyme-linked immunosorbent assay (ELISA) or Western blotting.

2. The method according to claim 1, wherein the amount of interleukin-6 is 10 pg/mL or more.

3. A diagnostic kit for acute myocardial infarction comprising an antibody specifically binding to cholesteryl ester transfer protein (CETP) or apoC-□.

4. The diagnostic kit ac cording to claim 3, wherein the cholesteryl ester transfer protein is detected by using a diagnostic tool comprising enzyme-linked immunosorbent assay (ELISA) or Western blotting.

5. A method of diagnosing acute myocardial infarction comprising measuring a triglyceride level in a high-density lipoprotein 2 (HDL₂) fraction of a serum specimen in the range of 60 to 80 mg/dL (0.39±0.05 mg/mg protein) by using a serum automated analyzer or a kit for detecting a triglyceride level.

6. The method according to claim 5, wherein the high-density lipoprotein 2 (HDL₂) fraction has a density higher than 1.063 g/mL and lower than 1.125 g/mL during the fractionation of lipoproteins in a serum.

7. A method of diagnosing acute myocardial infarction comprising measuring a triglyceride level in a low-density lipid fraction of a serum specimen is measured in the range of 185 to 305 mg/dL (0.27±0.06 mg/mg protein) by using a diagnostic tool including a serum automated analyzer or a kit for detecting a triglyceride level.

8. The method according to claim 7, wherein the low-density lipoprotein fraction has a density higher than 1.019 g/mL and lower than 1.063 g/mL during the fractionation of lipoproteins in a serum.

9. A method of diagnosing acute myocardial infarction comprising measuring a cholesterol level in a high-density lipoprotein 2 (HDL₂) fraction of a serum specimen is measured in the range of 46 to 58 mg/dL (0.28±0.03 mg/mg protein) by using a diagnostic tool including a serum automated analyzer or a kit for detecting a cholesterol level.

10. The method according to claim 9, wherein the high-density lipoprotein 2 (HDL₂) fraction has a density higher than 1.063 g/mL and lower than 1.125 g/mL during the fractionation of lipoproteins in a serum.
